(19) United States
(12) Patent Application Publication
(10) Pub. No.: US 2012/0214710 A1
(43) Pub. Date: Aug. 23, 2012

(54) METHOD OF PREDICTING RESPONSE TO THALIDOMIDE IN MULTIPLE MYELOMA PATIENTS

(75) Inventors: Rajesh Rajpal, Dublin (IE); Paul Dowling, Dublin (IE); Martin M. Clynes, Dublin (IE); Peter O'Gorman, Dublin (IE); Colin Clarke, Waterford (IE)

(73) Assignee: Dublin City University, Dublin (IE)

(21) Appl. No.: 13/390,836

(22) PCT Filed: Aug. 17, 2010
(86) PCT No.: PCT/EP2010/061995

§ 371 Date: Apr. 26, 2012

(30) Foreign Application Priority Data
Aug. 17, 2009 (GB) 0914330.6

(51) Int. Cl. G01N 33/574; C40B 40/10

(52) U.S. Cl. 506/18; 436/501; 435/7.92

(57) ABSTRACT

A method of predicting response to thalidomide, or thalidomide analogs, in an individual with cancer, especially cancers for which thalidomide has been implicated as a treatment, such as Multiple Myeloma (MM) employs one or more of a panel of biomarkers that have been shown to be differentially expressed in cancer patients that respond to thalidomide (hereafter “Responders”) relative to cancer patients that do not respond to thalidomide (hereafter “Non-responders). The method involves assaying a biological sample from the individual to determine the abundance of at least three biomarkers including Vitamin-D binding protein precursor (VDB) (Sequence ID 1) and Serum amyloid A protein (SAA) (Sequence ID 3), and at least one of beta-2-microglobulin (B2M) (Sequence ID 4), Haptoglobin (Hp) precursor (fragment) (Sequence ID 5), and zinc-alpha-2-glycoprotein (ZAG) (Sequence ID 2). Correlation of the abundance value for the at least three biomarkers with a reference abundance value from a Responder or Non-responder enables predication of response to thalidomide for the patient.

### TECHNICAL FIELD

The invention relates to a method of predicting response to thalidomide in a multiple myeloma patient.

### BACKGROUND TO THE INVENTION

Multiple Myeloma (MM) is a disease characterized by a proliferation of malignant plasma cells and a subsequent overabundance of monoclonal para-protein. Although Multiple Myeloma remains an incurable blood cancer the development of novel therapies has dramatically increased response rates and survival in the last 3 years. Despite major advances in our understanding of this complex disease a standard remission-induction therapeutic approach is taken to patients in similar categories of age and performance status in the great majority of centres. High dose chemotherapy with autologus stem cell transplant remains the standard therapy for younger patients (<65 yrs).

Thalidomide is an oral drug that has been shown to be highly active against myeloma with the respond rate of range from different trials. Serious side effects observed with use of thalidomide in myeloma include thrombo-embolic disease and peripheral neuropathy. Neuropathy is irreversible and may be disabling if not detected early and drug stopped. Significant Thalidomide-related neuropathy can preclude the subsequent use of other potentially neurotoxic agents such as Bortezomib.

### BRIEF DESCRIPTION OF THE INVENTION

Briefly, the invention provides a method of predicting response to thalidomide, or thalidomide analogs, in an individual with cancer, especially cancers for which thalidomide has been implicated as a treatment, such as Multiple Myeloma (MM). The method employs one or more of a panel of biomarkers that have been shown to be differentially expressed in cancer patients that respond to thalidomide (hereafter “Responders”) relative to cancer patients that do not respond to thalidomide (hereafter “Non-responders). The method involves assaying a biological sample from the individual to determine the abundance of at least one biomarker selected from the group consisting of: Vitamin-D binding protein precursor (VDB) (Sequence ID 1); zinc-alpha-2-glycoprotein (ZAG) (Sequence ID 2); Serum amyloid A protein (SAA) (Sequence ID 3); beta-2-microglobulin (B2M) (Sequence ID 4); and Haptoglobin (Hp) precursor (fragment) (Sequence ID 5). Correlation of the abundance value for the at least one biomarker with a reference abundance value from a Responder or Non-responder enables predication of response to thalidomide for the patient.

Preferably, the abundance value(s) from the individual are correlated with reference abundance value(s) from a Responder.

The term “Responders” should be understood to include individuals that demonstrate both complete response (CR) and those that demonstrate very good partial response (VGPR). The individual may be a human or a higher mammal, and may be Caucasian, and of European, Celtic and/or US origin.

Thus, for the biomarkers VDB, ZAG, TYR, SAA, a significant increase in abundance of the biomarker relative to the level of expression of the biomarker in a Responder indicates that the patient is a Non-responder. For VDB and the cohort of patients chosen, a significant increase in abundance preferably means a ratio of sample abundance to reference Responder abundance of 1.31 (2D-DIGE) or greater, or 1.28 (ELISA) or greater (See Table 1). For the biomarker Hp and the cohort of patients chosen, a significant decrease in abundance of Hp relative to the abundance of Hp in a Responder indicates that the patient is a Non-responder. For Hp, a significant decrease in abundance preferably means a ratio of sample abundance to reference Responder abundance of −3.01 (2D-DIGE) or greater or −1.73 (ELISA) or greater (See Table 1). For the biomarker ZAG and the cohort of patients chosen, a significant increase in abundance of ZAG relative to the abundance of ZAG in a Responder indicates that the patient is a Non-responder. For ZAG, a significant increase in abundance preferably means a ratio of sample abundance to reference Responder abundance of 1.48 (2D-DIGE) or greater or 1.27 (ELISA) or greater (See Table 1). For the biomarker B2M and the cohort of patients chosen, a significant increase in abundance of B2M relative to the abundance of B2M in a Responder indicates that the patient is a Non-responder. For B2M, a significant increase in abundance preferably means a ratio of sample abundance to reference Responder abundance of 1.96 (2D-DIGE) or greater or 2.00 (ELISA) or greater (See Table 1). For the biomarker SAA and the cohort of patients chosen, a significant increase in abundance of SAA relative to the abundance of SAA in a Responder indicates that the patient is a Non-responder. For SAA, a significant increase in abundance preferably means a ratio of sample abundance to reference Responder abundance of 3.01 (2D-DIGE) or greater or 3.80 (ELISA) or greater (See Table 1).

It will be appreciated that the determination of a significant difference between sample and reference values for any biomarker may vary from population to population, due to genetic heterogenicity, and also due to differing methods of detection. However, the invention is not restricted to any specific reference values for a given biomarkers, but is based on the detection of clinically significant modulation of abundance of biomarkers between a sample and reference values, be they reference values from Responders or Non-responders.

In a preferred embodiment, the abundance of at least two biomarkers is determined. Examples of combinations of two biomarkers include: SAA+Hp; SAA+VDB; Hp+VDB; SAA+B2M; Hp+B2M; VDB+B2M.

In a more preferred embodiment, the abundance of at least three biomarkers is determined. Examples of combinations of three biomarkers include: SAA+Hp+VDB; SAA+VDB+B2M; Hp+VDB+B2M; and SAA+B2M+Hp. In one embodiment, the combination of three biomarkers comprises two of SAA, Hp and VDB, and one selected from ZAG, TYR, and B2M, for example SAA+Hp+ZAG, SAA+VDB+ZAG, etc.

In a more preferred embodiment, the abundance of at least four biomarkers is determined. Examples of combinations of four biomarkers include: SAA+Hp+VDB+ZAG; SAA+Hp+VDB+B2M; Hp+VDB+B2M+ZAG; and SAA+B2M+Hp+ZAG. In one embodiment, the combination of four biomarkers comprises SAA, Hp and VDB, and one selected from ZAG, TYR, and B2M, for example SAA+Hp+VDB+ZAG, SAA+Hp+VDB+B2M, SAA+Hp+VDB+TYR, etc.

In a more preferred embodiment, the abundance of at least five biomarkers is determined. Examples of combinations of five biomarkers include: SAA+Hp+VDB+ZAG+B2M; SAA+Hp+VDB+ZAG+TYR; SAA+Hp+VDB+B2M+TYR; Hp+VDB+B2M+ZAG+TYR; and SAA+B2M+Hp+ZAG+TYR. In one embodiment, the combination of four biomarkers comprises SAA, Hp, VDB and ZAG, and one selected from TYR, and B2M, for example SAA+Hp+VDB+ZAG+TYR, or SAA+Hp+VDB+ZAG+B2M.

In another embodiment, the abundance of the six biomarkers SAA+Hp+VDB+ZAG+B2M+TYR is determined.

In a particularly preferred embodiment of the invention, the invention involves determining modulated abundance of at least three biomarkers comprising SAA and VDB, and at least one of ZAG, Hp and B2M.

In one embodiment, the at least three biomarkers comprise SAA, VDB and ZAG, and optionally one or more biomarkers selected from B2M and Hp (for example SAA+VDB+ZAG+B2M or SAA+VDB+ZAG+Hp).

In another embodiment, the at least three biomarkers comprise SAA, VDB and Hp, and optionally one or more biomarkers selected from ZAG and B2M (for example SAA+VDB+Hp+B2M).

In another embodiment, the at least three biomarkers comprise SAA, VDB and B2M, and optionally one or more biomarkers selected from ZAG and Hp.

The invention also provides a kit of parts comprising diagnostic reagents capable of quantitative detection of a panel of biomarkers comprising least two biomarkers selected form the group consisting of: VDB, ZAG, TYR, SAA, B2M, and Hp, and instructions for the use of the reagents in determining the response of an individual with cancer, especially multiple myeloma, to thalidomide. Suitably, the kit comprises or consist essentially of diagnostic reagents capable of quantitative detection of 3, 4, 5 or 6 biomarkers. In a particularly preferred embodiment, the panel of biomarkers comprises at least three biomarkers comprising SAA and VDB, and optionally one or more biomarkers selected from ZAG, Hp and B2M. In one embodiment, the panel of biomarkers comprises or consists essentially of SAA, VDB and ZAG, and optionally one or more biomarkers selected from B2M and Hp (for example SAA+VDB+ZAG+B2M or SAA+VDB+ZAG+Hp). In another embodiment, the panel of biomarkers comprises or consists essentially of SAA, VDB and Hp, and optionally one or more biomarkers selected from ZAG and B2M (for example SAA+VDB+Hp+B2M). In another embodiment, the panel of biomarkers comprises or consist essentially of SAA, VDB and B2M, and optionally one or more biomarkers selected from ZAG and Hp. The or each diagnostic reagent is typically an antibody, or antibody fragment, capable of specifically binding to the target biomarker. Suitably, the kit is an ELISA immunoassay.

Thus, in one specific embodiment, the invention relates to an immunoassay kit comprising a support having affixed thereon an antibodies, or antibody fragments, capable of specifically binding to a panel of biomarkers comprising at least 3, 4, 5 or 6 biomarker proteins selected from the group consisting of VDB, ZAG, TYR, SAA, B2M, and Hp, and means for quantitatively detecting specific binding between the antibodies, or antibody fragments, and biomarkers proteins. Preferably, the immunoassay kit is adapted for the specific detection of a panel of biomarkers comprising SAA and VDB and one or more of ZAG, Hp and B2M. In one embodiment, the immunoassay kit is adapted for the specific detection of a panel of biomarkers comprising or consisting essentially of SAA, VDB and ZAG, and optionally one or more biomarkers selected from B2M and Hp (for example SAA+VDB+ZAG+B2M or SAA+VDB+ZAG+Hp). In another embodiment, the immunoassay kit is adapted for the specific detection of a panel of biomarkers comprising or consisting essentially of SAA, VDB and Hp, and optionally one or more biomarkers selected from ZAG and B2M (for example SAA+VDB+Hp+B2M). In another embodiment, the immunoassay kit is adapted for the specific detection of a panel of biomarkers comprising or consisting essentially of SAA, VDB and B2M, and optionally one or more biomarkers selected from ZAG and Hp.

The invention also relates to a method of treating an individual with a cancer of the type that is responsive to thalidomide, comprising a step of predicting the individuals response to thalidomide using the method of the invention, and when the individual is predicted to be a Responder, treating the individual with thalidomide, or when the individual is predicted to be a Non-response, treating the individual with a non-thalidomide therapy.

In the methods of the invention, differential abundance may be determined by performing the assay in tandem with a reference sample (or samples) from patients known to be Responders, or with a reference sample (or samples) from patients known to be Non-responders). Generally differential expression is detected by comparing a value for one or more of the biomarkers from the patient sample with the value determined from the reference sample. In an alternative embodiment, the method may be performed by detecting absolute expression levels of one or more of the biomarkers from the patient sample, for example by quantitative ELISA, and comparing the value obtained with known values from Responders (or Non-responders) to detect differential expression. Table 3 below provides mean values for the biomarkers SAA, ZAG, VDB, Hp and B2M (μg/ml) for Responders and Non-responders. It will be appreciated that for different populations, the reference values (or cut-off values) may vary due to various factors, including population genetic differences. Correlating the differential abundance for a combination of biomarkers, for example SAA, VDB and SAA, with response can be carried out using a number of different statistical techniques. An example of a suitable algorithm is provided below.

Generally speaking, the biomarker is a protein. However, the method of the invention may also be performed by detecting differential expression by other means, for example, the enumeration of mRNA copy number.

Generally speaking, the biological sample is a blood sample, especially blood serum or plasma. However, other biological samples may also be employed, for example, cerebrospinal fluid, saliva, urine, or cell or tissue extracts.

Generally speaking, the individual is a human, although the method of the invention is applicable to other higher mammals. The invention is especially useful in predicting response to thalidomide in newly diagnosed individuals, but is also applicable to patients that have established primary disease, and those with relapsed or refractory cancer.

In this specification, the term “cancer” should be understood to mean a cancer that is responsive to thalidomide treatment in at least part of the population. An example of such a cancer is a haematological malignancy such as multiple myeloma, prostate cancer, glioblastoma, and lymphoma. Other cancers potentially responsive to thalidomide include: fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias.

In this specification, the term “thalidomide” should be understood to mean drugs or pharmaceutical formulations comprising or consisting of the active thalidomide compound 2-(2,6-dioxopiperidin-3-yl)-1H-isoindole-1,3(2H)-dione. The term “thalidomide analogs” should be understood to mean close structural variants of thalidomide that have a similar biological activity such as, for example, lenalidomide (REVLIMID) ACTIMID™ (Celgene Corporation), and the compounds disclosed in U.S. Pat. No. 5,712,291, W002068414, and W02008154252 (the complete contents of which are incorporated herein by reference).

In this specification, the term “antibody” should be understood to mean an intact immunoglobulin or to a monoclonal or polyclonal antigen-binding fragment with the Fc (crystallizable fragment) region or FcRn binding fragment of the Fc region, referred to herein as the “Fc fragment” or “Fc domain”, which has a binding affinity for the target biomarker. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the biomarker polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies.

An immunoglobulin is typically a tetrameric molecule. As used herein, the term “immunoglobulin” refers to one or more chains of the tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. In human, there are in addition four IgG (IgG1, IgG2, IgG3 and IgG4) and two IgA subtypes present. The variable regions of each light/heavy chain pair form the antibody binding site such that an intact natural immunoglobulin has two binding sites.

In this specification, the term “antibody fragment” should be understood to mean a single chain (sc) Fv or Fab fragment domain antibody that bind a biomarker of the claimed invention. Antibody fragments are produced by means of amplification in a suitable producer cell, for example Escherichia coli. An scFv antibody fragment is such that wherein the light chain variable region and heavy chain variable region are connected in series in a single molecule, usually by means of a linker. In this specification, the term “immunodetection” should be understood to mean an antibody labeled to facilitate detection. That is, where another molecule is incorporated in the antibody, for example, incorporation of a radiolabeled amino acid. Various methods of labeling are known in the art and may be used, for example, radioisotopes or radionuclides (e.g., ³H, ¹⁴C, ¹⁵N, ³⁵S), fluorescent labels (e.g., FITC, rhodamine, lanthanide phosphors), enzymatic labels (e.g., horseradish peroxidase, beta-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, and toxins such as pertussis toxin, ethidium bromide, etoposide, vincristine, colchicin, doxorubicin, daunorubicin, dihydroxy anthracin dione, mitoxantrone, mithramycin, actinomycin D, 1-dehydrotestosterone, and analogs or homologs thereof. Procedures to detect such the binding of a labelled antibody to a target protein are well known to those skilled in the art, for example, western blotting, Enzyme-linked Immunosorbent Assay (ELISA), immunofluorescence microscopy, magnetic immunoassay, and radioimmnuassay, and the like.

### BRIEF DESCRIPTION OF THE FIGURES

FIG. 1

Statistical analysis of Hp & B2M respectively, using DeCyder BVA software. A & B display that these proteins were found to be decreased and increased respectively, in the immunodepleted serum from non-responders compared to responders. C & D also show gel images and 3-D views for Hp & B2M respectively, showing a clear change in expression levels. B2M: beta-2-microglobulin, Hp: Haptoglobin.

FIG. 2

Displayed are the concentrations for the five differentially expressed proteins, obtained in duplicate for each patient using ELISAs. The box plots show the data for responder and non-responder patients. The horizontal lines within the boxes represent the median. The upper and lower box edges are the 1st and 3rd quartiles. The whiskers reach the nearest value within 1.5 times the inter quartile range. The points outside the whiskers are considered outliers. A. B2M: beta-2-microglobulin, B. VDB: Vitamin D binding protein, C. ZAG: Zinc alpha 2-glycoprotein, D. Hp: Haptoglobin, E. SAA: Serum Amyloid A protein.

FIG. 3

Logistic regression analysis used to develop a predictive model for each individual differentially expressed protein. The performance of the models was assessed using ROC curves (A), and the AUC for each individual protein are shown (B). The best predictive ability for logistic regression model for single proteins was for B2M and SAA, with AUC values of 0.87 and 0.82, respectively. ZAG: Zinc alpha 2-glycoprotein, VDB: Vitamin D binding protein, SAA: Serum Amyloid A protein, B2M: beta-2-microglobulin, Hp: Haptoglobin.

FIG. 4

ROC curve analysis using a combination of Hp, SAA and VDB. The best possible AUC was found with combined Hp, SAA and VDB, which yielded an AUC of 0.96 indicating excellent discriminatory power. Hp: Haptoglobin, SAA: Serum Amyloid A protein, VDB: Vitamin D binding protein.

### DETAILED DESCRIPTION

Patients & Sample Collection:

Serum samples from 51 consecutive newly diagnosed MM patients who were having initial treatment with thalidomide-based regimens were analyzed. Samples were obtained at diagnosis and prior to commencement of therapy. The samples were collected according to standard phlebotomy procedures from consented patients. Ethical consent was granted from the Mater Misericordiae University Hospital, Dublin, Ireland ethics committee. 10 ml of blood sample was collected into additive free blood tubes and was allowed to clot for 30 minutes to 1 hour at room temperature. Samples were coded and transported on ice to the laboratory. The serum was denuded by pipette from the clot and place into a clean tube. The tubes were centrifuged at 400 relative centrifugal force (rcf) for 30 minutes at 4° C. Serum was aliquoted in the cryovial tubes, labeled and stored at −80° C. until time of analysis. The time from sample procurement to storage at −80° C. was less than 3 hours. Each serum sample underwent no more than 3 freeze/thaw cycles prior to analysis.

Removal of High Abundance Proteins from Serum Samples:

Samples were prepared as outlined previously (Dowling P, O'Driscoll L, Meleady P, et al. Electrophoresis. 2007; 28:4302-4310). Briefly, diluted samples (Buffer A) were centrifuged to remove lipids and particulates. The Human Multiple Affinity Removal System (MARS) column was employed to remove 14 of the most abundant proteins (albumin, IgG, antitrypsin, IgA, transferrin, haptoglobin, fibrinogen, alpha2-macroglobulin, alpha1-acid glycoprotein, IgM, apolipoprotein AI, apolipoprotein AII, complement C3 and transthyretin). For each sample, a low abundance fraction was collected and concentrated using 5 kDa molecular weight cut-off spin concentrators (Agilent Technologies). The concentrated depleted serum samples were collected and immediately transferred and stored at −80° C. until further analysis. The depletion protocol was found to be reproducible as demonstrated by Western Blot analysis using an anti-albumin antibody, which confirmed the absence of albumin from the immunodepleted fraction (results not shown).

Preparation for 2-D Electrophoresis:

Proteins from the immunodepleted serum samples were precipitated prior to labeling using a 2-D Cleanup Kit (Biorad). The protein pellets were resuspended in ice-cold DIGE lysis buffer [20 mM Tris, 7 M Urea, 2 M Thiourea, 4% CHAPS pH 8.5]. Protein quantification was performed using the Quick Start Bradford Protein Assay (Biorad) absorbance at 595 nm using bovine serum albumin as a protein standard (Dowling P, O'Driscoll L, Meleady P, et al. Electrophoresis. 2007; 28:4302-4310).

2D-Difference Gel Electrophoresis (2D-DIGE) Labeling & Running:

Depleted serum samples were labeled with N-hydroxy succinimidyl ester-derivatives of the cyanine dyes Cy2, Cy3, and Cy5 following a standard protocol (Dowling P, O'Driscoll L, Meleady P, et al. Electrophoresis. 2007; 28:4302-4310). Immobilized 24 cm linear pH gradient (IPG) strips, pH 3-11NL, were rehydrated in rehydration buffer (7M Urea, 2M Thiourea, 4% CHAPS, 0.5% IPG Buffer, 50 mM DTT) overnight, according to the standard guidelines (Dowling P, O'Driscoll L, Meleady P, et al. Electrophoresis. 2007; 28:4302-4310; and Nagalla S R, Canick J A, Jacob T, et al. J Proteome Res. 2007; 6:1245-1257). Iso-Electric-Focusing (IEF) was performed using an IPGphor apparatus (GE Healthcare) for a total of 40 kV/h at 20° C. Equilibrated IPG strips were transferred onto 12.5% uniform polyacrylamide gels poured between low fluorescence glass plates. Strips were overlaid with 0.5% w/v low-melting-point agarose in running buffer containing bromphenol blue. Gels were run using the Ettan Dalt 12 apparatus (GE Healthcare) at 2.5 W/gel for 30 minutes then 100 W in total at 10° C. until the dye front had run off the bottom of the gels.

DeCyder Analysis:

All of the gels were scanned using the Typhoon 9400 Variable Mode Imager (GE Healthcare) to generate gel images at the appropriate excitation and emission wavelengths from the Cy2, Cy3 and Cy5 labeled samples. The resultant gel images were cropped using the Image Quant software tool and imported into Decyder 6.5 software. The biological variation analysis (BVA) module of Decyder 6.5 was used to compare the immunodepleted serum from responders versus non-responders to generate lists of differentially expressed proteins (Dowling P, O'Driscoll L, Meleady P, et al. Electrophoresis. 2007; 28:4302-4310; and Huang J T, Wang L, Prabakaran S, et al. Mol Psychiatry. 2008; 13:1118-1128). The differential in gel analysis module was used to assign spot boundaries and to calculate parameters such as normalized spot volume. The BVA mode of DeCyder 6.5 was then used to match all pair use image comparisons from difference in-gel analysis for a comparative cross gel statistical analysis. At this stage operator intervention was required for the more accurate matching. If the matching in an area required correction, the current matches were broken and remade with the appropriate spots (Dowling P, O'Driscoll L, Meleady P, et al. Electrophoresis. 2007; 28:4302-4310; and Nagalla S R, Canick J A, Jacob T, et al. J Proteome Res. 2007; 6:1245-1257).

Spot Digestion and Identification by Mass Spectrometry Analysis:

Proteins of interest were robotically picked from preparative gels containing 400 mg of protein stained with Colloidal Coomassie Blue (CBB) stain (Sigma) using the Ettan Spot Picker robot (GE Healthcare). Tryptic digestions were performed on the proteins of interest according to standard protocols (Dowling P, O'Driscoll L, Meleady P, et al. Electrophoresis. 2007; 28:4302-4310). Tryptic digested proteins were analyzed by one-dimensional LC-MS using the Ettan MDLC system (GE Healthcare) in high-throughput configuration directly connected to a Finnegan LTQ (Thermo Electron). Samples were concentrated and desalted on RPC trap columns (Zorbax 300S B C18, 0.3 mm×5 mm, Agilent Technologies) and the peptides were separated on a nano-RPC column (Zorbax 300S B C18, 0.075 mm×100 mm, Agilent Technologies) using a linear acetonitrile gradient from 0% to 65% ACN (Sigma) over 60 minutes. All buffers used for nano LC separation contained 0.1% formic acid (Fluka) as the ion pairing reagent (Dowling P, Wormald R, Meleady P, Henry M, Curran A, Clynes M. J Proteomics. 2008; 71:168-175)

Protein identification was performed using the Turbo-SEQUEST algorithm in the BioWorks 3.1 software package (Thermo Electron) and the Swiss-Prot human database (Swiss Institute of Bioinformatics, Geneva, Switzerland). The identified peptides were further evaluated using charge state versus cross-correlation number (XCorr). The criteria for positive identification of peptides was XCorr>1.5 for singly charged ions, XCorr>2.0 for doubly charged ions, and XCorr>2.5 for triply charged ions, together with a minimum of 2 matched peptides for each protein.

Enzyme Linked Immunosorbent Assay (ELISA):

ELISAs were used to confirm the differential expression of the six potential biomarkers discovered using 2D-DIGE analysis. ELISA-based validation was carried out using raw unfractionated serum samples from the original cohort of patients. Each sample was analyzed in duplicate using the following commercially available kits, for the measurement of human serum amyloid A (Invitrogen), serum haptoglobin (AssayMax), zinc-alpha-2-glycoprotein (Bio-Vendor), beta-2-microglobulin and vitamin D binding protein (Immunodiagnostic) kits were used. The ELISA assays were performed according to each manufacturer's protocol and guidelines. The optical density (OD) was measured using a micro-plate reader (Bio-Tek) and the concentration of each protein in the serum samples were determined by comparing the OD of the samples against the respective standard curve provided by the kit.

Statistical Analysis:

DIGE gels were exported for image analysis using the Biological Variation Analysis (BVA) module of Decyder 6.5 software (GE Healthcare) for quantitation of protein abundance levels. Following confirmation of appropriate spot detection, matching, normalization and spot statistics were reviewed. The normalized volume of a spot was compared in all the gels between each group. Spots that were found to be statistically significant (t-test≦0.01) were selected for further analysis.

Logistic regression and ROC curve analysis were carried out in the freely available statistical software R (http://www.r-project.org/). The ROC curves were used to interpret the utility of logistic regression models for various combinations of the differentially expressed (DE) proteins. The probability of correct prediction for a given model was calculated from the ROC curve by determining the area under curve (AUC). Thus the proteins and combinations of proteins returning the largest AUC values are deemed the most effective for the discrimination of responders from non-responders (Fu A Z, Cantor S B, Kattan M W. Use of Nomograms for Personalized Decision-Analytic Recommendations. Med Decis Making. 2009).

Akaike's Information Criterion (AIC) was also used as a criterion to select the best combination of biomarkers. AIC is a commonly used measure to select between competing statistical models. The AIC is a tradeoff between goodness of fit and model complexity i.e. the number of parameters required (in this case proteins).

Sensitivity and specificity values were calculated for the best combination of biomarkers. Sensitivity is defined as the percentage of all non-responders who were refractory to a thalidomide-based treatment regime correctly identified as having this phenotype based on the panel of protein biomarkers (the true positive rate). Specificity is defined as the percentage of all responders who were sensitive to a thalidomide-based treatment regime correctly identified as having this phenotype based on the panel of protein biomarkers (the true negative rate).

As an additional measure of the predictive potential of these biomarkers to accurately predict response to thalidomide-based therapy, a commonly used internal validation technique known as LOOCV was performed. During the LOOCV procedure data from a single observation is removed from the dataset and the remaining samples are then utilized to construct a logistic regression model. The “test” sample is presented to the trained model and the performance assessed, LOOCV continues until each observation is designated as the “test”. The average performance over the 51 tests is reported as the LOOCV accuracy.

Results:

Sample Set:

The mean age of the patient group was 68 SD+/−6.95 years (range 52-81 years); 27 were male and 24 female. Based on Day-100 re-staging investigations and using the International Myeloma Working Group (IMWG) uniform response criteria (Rajkumar S V, Buadi F. Multiple myeloma: new staging systems for diagnosis, prognosis and response evaluation. Best Pract Res Clin Haematol. 2007; 20:665-680; Durie B G, Harousseau J L, Miguel J S, et al. International uniform response criteria for multiple myeloma. Leukemia. 2006;20:1467-1473) for MM, 29 responders and 22 non-responders to thalidomide-based therapy were identified. The mean age was 66 SD+/−6.80 years (range, 52-81 years) for responders and 70 SD+/−6.69 years (range, 57-79 years) for non-responders. Based on the ISS classification (Hotta T. [Classification, staging and prognostic indices for multiple myeloma]. Nippon Rinsho. 2007; 65:2161-2166), 6 responding patients had stage I, 15 had stage II and 8 had stage III disease. Two responders had stage I, 11 had stage II and 9 had stage III disease. In the responders group, 24 patients were treated with thalidomide and dexamethasone (TD), 3 with thalidomide, cyclophosphamide and dexamethasone (CTD) and 2 with melphalan, prednisone and thalidomide (MPT). In the non-responder group, 17 patients received TD, 4 received CTD and 1 patient received MPT. Median follow-up was 13 months (range, 6-21 months). In the responder group, 9 patients achieved complete response (CR) and 20 achieved very good partial response (VGPR). Five non-responders had Stable disease (SD) and the remaining 17 had Progressive disease (PD) (Table I).

Proteomic Profiling:

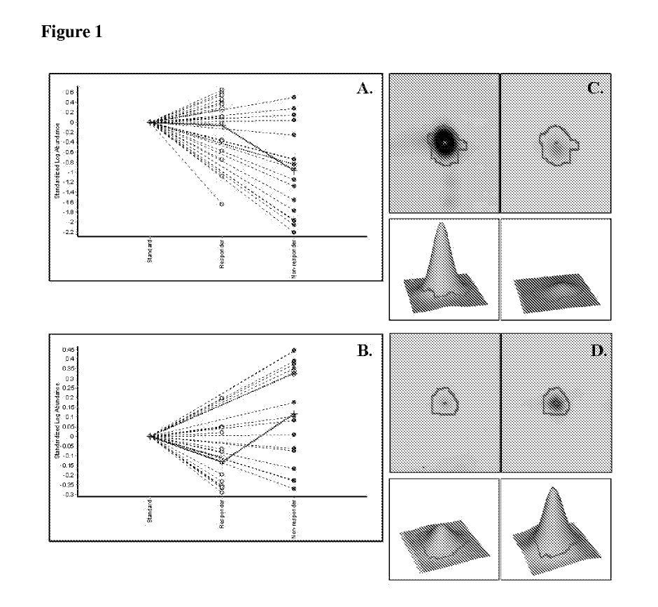

Proteins were precipitated from the low abundance immunodepleted fraction, resuspended in lysis buffer, fluorescently labeled, and analyzed by 2D-DIGE using an internal standard design. This analysis was performed on 39 newly diagnosed MM patients (22 responders; 17 non-responders). Spot maps were generated and maps were aligned with a master spot map; relative abundance values were generated for each of 886 protein spots that were common to more than 90 percent of gels. Based on 2D-DIGE analysis, protein spots with a fold change of ≧1.25 in abundance level and a t-test of ≦0.01 were selected. Using these criteria, five individual differentially expressed proteins spots were detected. Four proteins were increased in abundance level, and one was decreased in abundance level in thalidomide non-responders compared to responders. FIG. 1 shows DeCyder analysis for B2M & Hp, indicating that these proteins were increased and decreased in abundance levels respectively, between non-responders and responders to thalidomide based therapy. Gel images and 3-D protein spot views for Hp & B2M are also displayed, demonstrating a clear difference in the abundance levels (FIG. 1).

Protein Identification:

Subsequently, these proteins of interest were identified by LC-MS/MS using an ion trap LTQ mass spectrometer and searched against the SWISS PROT database using SEQUEST. The serum Haptoglobin fragment (Hp), which was the only protein found to have a lower abundance level in non-responders compared to responders, was identified by LC-MS/MS resulting in 9 matched peptide corresponding to a sequence coverage of 11.58 percentage (%) (Table II). Proteins found to have higher abundance level between non-responders and responders to thalidomide based therapy were Zinc alpha 2-glycoprotein (ZAG), Vitamin D binding protein (VDB), Serum Amyloid A protein (SAA), and beta-2-microglobulin (B2M). These proteins were identified by LC-MS/MS resulting in 10, 20, 4, and 3 matched peptides respectively, corresponding to a 37.63, 53.59, 48.36 and 35.29 percentage (%) sequence coverage, respectively (Table II).

DeCyder Ratios, ELISA Data and ROC Curve Analysis:

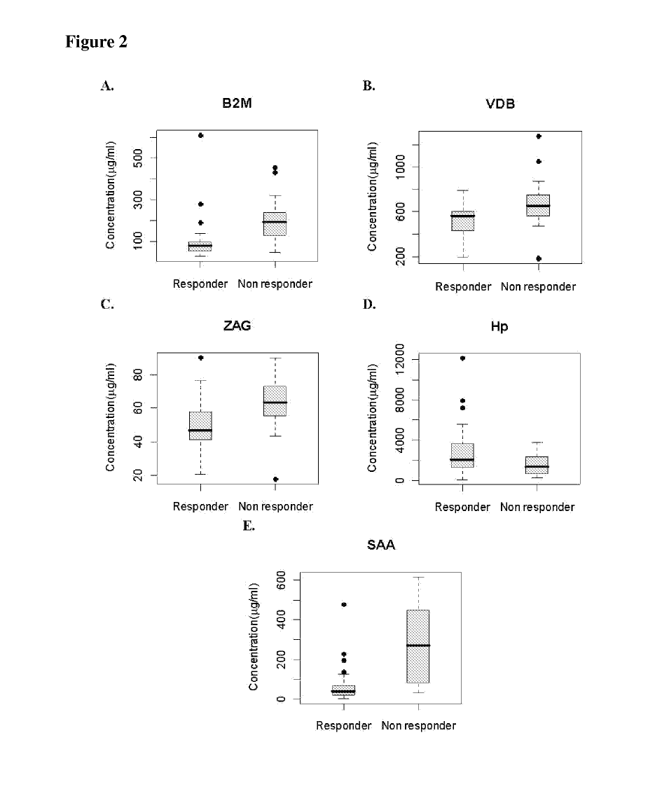

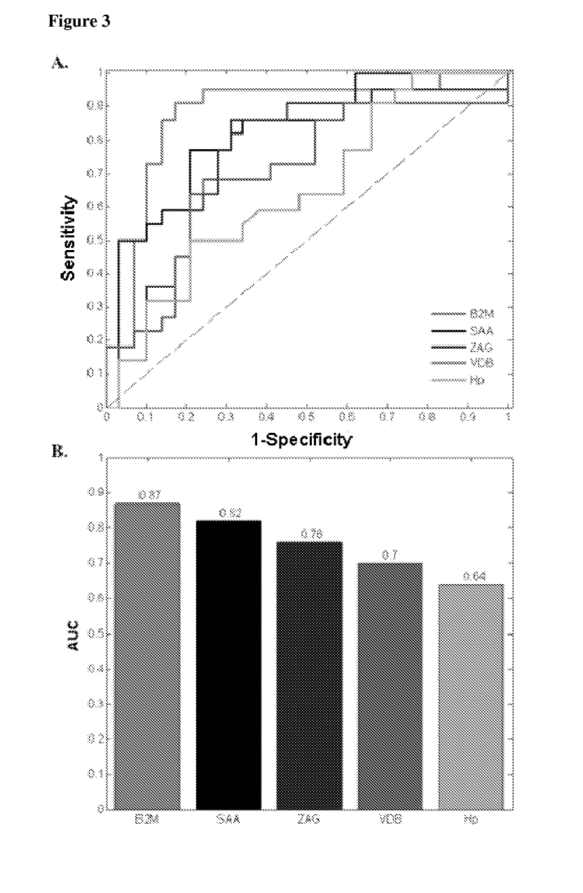

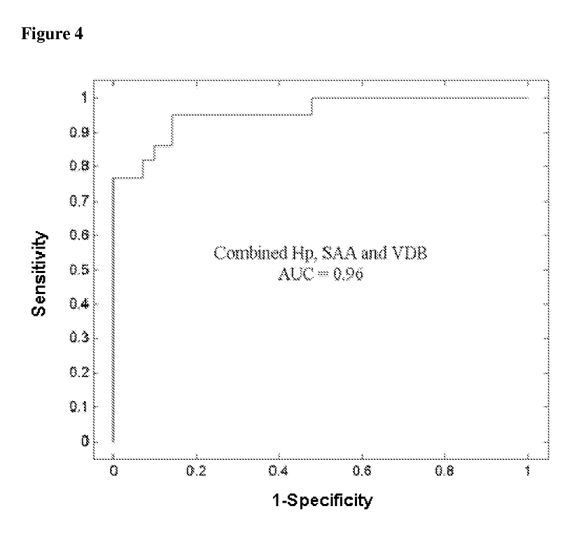

In this study, ELISA-based assays were used to measure the levels of the five candidate marker proteins in serum from thalidomide responders and non-responders (Table III). The ELISA-based assays were performed on a larger cohort compared to the 2D-DIGE analysis, consisting of 51 consecutive MM patients (29 responders; 22 non-responders). The 5 differentially expressed protein concentrations were measured in duplicate for each patient. The box plots show the data for responders and non-responders (FIG. 2). The horizontal line within the boxes represents the median. The upper and lower box edges are the 1st and 3rd quartiles. The whiskers reach the nearest value within 1.5 times the inter-quartile range. The points outside the whiskers are considered outliers; however, no outlier value was removed from our analysis.

In clinical practice, it is rare that a chosen cut off point for a single analyte will achieve perfect discrimination between various groups of patients, and one has to select the best compromise between sensitivity and specificity by comparing the diagnostic performance of different tests or diagnostic criteria available. Accordingly, the suitability of a panel of proteins for potential clinical application was assessed using logistic regression ROC curves to report the AUC of each test. ROC curves allow systematic analysis of the diagnostic performance of a test, a comparison of the performance of different tests, and the AUC provides a summary measure of the utility of the model. The potential impact of the use of ZAG, SAA, VDB, B2M and Hp as single or combination biomarkers for distinguishing between responders and non-responders to thalidomide based therapy in MM patients were assessed. The ELISA values for each of these five proteins were used to develop LR models and the subsequent ROC curves generated (FIG. 3) and the AUC determined.

2D-DIGE and ELISA analysis showed that ZAG had a 1.48 (p=0.0000022) and a 1.27 (p=0.00398) fold increase in abundance levels in non-responders compared to responders, respectively (Table II). The ROC curve generated from the ELISA data for ZAG showed an AUC of 0.76 as an individual protein (FIG. 3). Results for SAA showed a 3.01 (p=0.006) fold increase in abundance levels for non-responders compared to responders using 2D-DIGE protein profiling analysis. This result correlated strongly with data from the ELISA analysis, indicating a 3.80 (p=0.00016) fold increase in SAA abundance levels in non-responders compared to responders (Table II). ROC curves calculated from this ELISA data showed an AUC of 0.82 (FIG. 3), indicating excellent discriminatory power for this single protein. SAA is a sensitive marker of inflammation. In the 22 non-responder patients studied in this project, none had evidence of infection or fever at the time of sampling; out of 29 responder patients, 1 had an infection resulting in mild fever. Therefore, the SAA elevation in the non-responder patients appeared to be unrelated to infection or inflammation.

Hp is normally removed by the immunodepletion column; however, using 2D-DIGE analysis followed by LC-MS/MS, a ˜10 kDa haptoglobin fragment was identified. The inventors suggest that this Hp fragment was not removed due to its size and non-interaction with the specific Hp antibody in the affinity column, and hence it was detected in the 2D-DIGE analysis. In the protein profiling analysis, the Hp fragment was found to be 3.01 (p=0.0015) fold decreased in serum from non-responders compared to responders. As intact Hp was removed from this analysis because of the immunodepletion column, it was decided to investigate this protein using an ELISA-based assay approach. Data from the ELISA analysis for intact Hp showed a 1.73 (p=0.03241) fold decrease in abundance levels in non-responders compared to responder. ROC curves were generated from the ELISA data which showed an AUC of 0.64 (FIG. 3). The decrease in abundance of the Hp fragment seen in thalidomide-non-responders compared to responders using 2D-DIGE analysis displayed a similar trend to that of the intact Hp detected by ELISA analysis in the unfractionated serum samples (Table II).

2D-DIGE data and ELISA results showed a 1.96 (p=0.0015) and 2.00 (p=0.00118) fold increase, respectively, in the abundance level of B2M from non-responders compared to responders. The ROC curve generated from this data showed an AUC of 0.87, indicating excellent discriminatory power for this protein (FIG. 3). The VDB protein level from 2D-DIGE analysis and ELISA data showed a 1.31 (p=0.00044) and 1.28 (p=0.02045) fold increase in abundance levels, respectively, from non-responders compared to responders (Table II). ROC curves were generated from the ELISA data and showed an AUC of 0.70 (FIG. 3).

Logistic Regression (LR):

Initially, LR was used to develop predictive models for each individual differentially expressed protein (FIG. 3). The best predictive single proteins from the LR model were B2M and SAA, with AUC values of 0.87 and 0.82, respectively. The remaining single protein model had AUC values less than 0.7, indicating poorer predictive ability (FIG. 3). The predictive capability of models developed based upon combinations of proteins was also assessed. LR models were constructed and ROC analysis carried out on all possible permutations of the differentially expressed proteins. Successful combinations of biomarkers from this analysis for predicting response to thalidomide based therapy, were found to be Hp, SAA, VDB (AUC=0.96, LOOCV=84.31, AIC=35.26), ZAG, B2M, SAA, VDB (AUC=0.96, LOOCV=84.31, AIC=37.02) and B2M, SAA, VDB (AUC=0.94, LOOCV=84.31, AIC=36.59). The combination of Hp, SAA, VDB was found to be successful based on the values from the AUC, LOOCV and AIC analyses as shown (FIG. 4). The sensitivity and specificity of this model was 81.81% and 86.20%, respectively. The combination of SAA, VDB and ZAG yielded an AUC of 0.96 indicating outstanding predictive capability.

In this analysis, CRP level at diagnosis was also assessed but did not improve the predictive capability of this model. CRP was found to have a mean of 10.4+/−17.7 μg/ml in responders compared to 12.1+/−23.6 μg/ml in non-responders (p=0.770749339). CRP was found to have an AUC of 0.4, indicating no discriminatory power for predicting response to thalidomide based therapy.

Calculation of Non-Response Probability:

The equation below allows for the calculation of the probability (p) of a patient being a non-responder based on the serum concentrations of ZAG, Hp and VDB as determined using ELISAs. Each protein concentration in μg/ml is first multiplied by the regression coefficient (derived from the fitted model) as per the equation below. If the resulting value of p is below 0.5, response to thalidomide is predicted and if the value is above 0.5 non-response to thalidomide is predicted.

$$p = \frac{1}{1 + {e}^{- ( ( - 0.001037 \times Hp ) + ( 0.017653 \times SAA ) + ( 0.013311 \times VDB ) - 8.603880 )}}$$

TABLE I

| | | | | | |
|---|---|---|---|---|---|
| | | | Day-100 | | |
| Patients | Patients | Clinical ‘ISS’ | IMWG | Response to | Follow |
| Age | Sex | Stage | re-staging | thalidomide | up-months |

Responders

| | | | | | |
|---|---|---|---|---|---|
| 73 | F | III | VGPR | R | 17 |
| 66 | M | II | CR | R | 16 |
| 58 | F | III | CR | R | 16 |
| 67 | M | II | VGPR | R | 12 |
| 59 | M | III | VGPR | R | 18 |
| 67 | M | III | CR | R | 16 |
| 72 | M | III | CR | R | 13 |
| 60 | M | II | CR | R | 19 |
| 71 | F | II | VGPR | R | 13 |
| 69 | M | I | VGPR | R | 12 |
| 61 | F | II | CR | R | 19 |
| 74 | F | I | VGPR | R | 13 |
| 70 | F | II | VGPR | R | 14 |
| 61 | F | I | VGPR | R | 12 |
| 77 | M | II | VGPR | R | 14 |
| 63 | M | I | CR | R | 14 |
| 58 | F | I | VGPR | R | 15 |
| 59 | F | I | VGPR | R | 14 |
| 64 | M | II | CR | R | 12 |
| 63 | F | II | VGPR | R | 21 |
| 65 | M | II | VGPR | R | 8 |
| 70 | M | III | VGPR | R | 7 |
| 66 | F | II | CR | R | 8 |
| 80 | M | II | VGPR | R | 7 |
| 66 | F | II | VGPR | R | 10 |
| 81 | F | III | VGPR | R | 6 |
| 52 | M | III | VGPR | R | 11 |
| 64 | M | II | VGPR | R | 6 |
| 68 | F | II | VGPR | R | 6 |

Non-Responders

| | | | | | |
|---|---|---|---|---|---|
| 72 | F | II | PD | NR | 16 |
| 74 | M | III | PD | NR | 12 |
| 71 | M | III | PD | NR | 19 |
| 65 | M | II | PD | NR | 11 |
| 70 | M | III | PD | NR | 17 |
| 63 | F | III | PD | NR | 14 |
| 72 | M | III | PD | NR | 16 |
| 63 | M | II | SD | NR | 11 |
| 78 | M | II | PD | NR | 10 |
| 79 | F | II | PD | NR | 15 |
| 57 | F | III | PD | NR | 10 |
| 77 | M | III | PD | NR | 12 |
| 78 | F | II | PD | NR | 15 |
| 64 | F | II | PD | NR | 21 |
| 65 | F | I | SD | NR | 20 |
| 73 | F | III | PD | NR | 18 |
| 78 | M | II | SD | NR | 11 |
| 61 | F | II | SD | NR | 12 |
| 76 | F | III | PD | NR | 9 |
| 77 | M | II | PD | NR | 11 |
| 62 | M | I | PD | NR | 8 |
| 69 | M | II | SD | NR | 6 |

Table I: This table outlines the clinical details for the patients included in this study, including their age, sex, ISS (International Staging System), Day 100 restaging results based on IMWG uniform response criteria for MM. The last column shows duration of follow-up in months.
Abbreviations;
CR: complete response,
VGPR: very good partial response,
SD: stable disease,
PD: Progressive disease,
IMWG: International Myeloma Working Group,
R: Responders to thalidomide-based therapy,
NR: Non-responders to thalidomide-based therapy,
M: male,
F: female.

TABLE II

| | | No. of Matched | | | DeCyder | | ELISA |
|---|---|---|---|---|---|---|---|
| Protein Name | M.W. (Da) | Peptides | % Coverage | DeCyder Ratio | p-value | ELISA Ratio | p-value |
| Vitamin D-binding protein (VDB) | 52930 | 20 | 53.59 | 1.31 | 0.00044 | 1.28 | 0.02045 |
| Haptoglobin fragment (Hp) | 45177 | 9 | 11.58 | −3.01 | 0.0017 | 1.73 | 0.03241 |
| Zinc-alpha-2-glycoprotein (ZAG) | 33851 | 10 | 37.63 | 1.48 | 0.0000022 | 1.27 | 0.00398 |
| Beta-2-microglobulin (B2M) | 13706 | 3 | 35.29 | 1.96 | 0.0015 | 2 | 0.00118 |
| Serum Amyloid A Protein (SAA) | 13524 | 4 | 48.36 | 3.01 | 0.006 | 3.8 | 0.00016 |

Table II: Listed are the protein identities obtained by LC-MS/MS analysis, molecular weight (M.W.), number of matched peptides related to the protein, % coverage of the protein sequence identified, DeCyder ratio with associated p-value (immunodepleted serum) and ELISA ratio (NR/R) with associated p-value (unfractionated serum).
The DeCyder ratio for Hp is the ~10 kDa fragment identified in the 2D-DIGE analysis while the ELISA ratio is based on the intact form of this protein.
LC-MS/MS: Liquid Chromatography Mass Spectrometry

TABLE III

| | | | | | |
|---|---|---|---|---|---|
| | B2M | VDB | ZAG | Hp | SAA |
| Samples | μg/ml | μg/ml | μg/ml | μg/ml | μg/ml |

Responders

| | | | | | |
|---|---|---|---|---|---|
| 1 | 83.7 | 587 | 46.9 | 473 | 4 |
| 2 | 47.5 | 429.6 | 76.6 | 1294 | 47.8 |
| 3 | 54.2 | 564.8 | 38.4 | 482 | 22.5 |
| 4 | 609.4 | 449.1 | 38.3 | 81 | 12.1 |
| 5 | 52.4 | 782.4 | 64.8 | 2121 | 45.6 |
| 6 | 79.2 | 765.7 | 67.8 | 5550 | 14.9 |
| 7 | 279.4 | 718.5 | 49.2 | 2044 | 66 |
| 8 | 92.4 | 750.9 | 55.1 | 692 | 11.9 |
| 9 | 63.8 | 596.3 | 61.3 | 7219 | 138.6 |
| 10 | 51.1 | 587 | 44 | 3378 | 196.3 |
| 11 | 98.1 | 525 | 42.5 | 1355 | 26.2 |
| 12 | 91.9 | 456.5 | 52.9 | 1746 | 15.2 |
| 13 | 99.4 | 604.6 | 58.1 | 3621 | 33.9 |
| 14 | 190.6 | 517.6 | 52.1 | 7939 | 26.1 |
| 15 | 46.7 | 449.1 | 43.5 | 1662 | 71.8 |
| 16 | 109.8 | 670.4 | 49.4 | 2676 | 5 |
| 17 | 72 | 410.2 | 67.7 | 4381 | 478.9 |
| 18 | 56.7 | 576.9 | 74.8 | 3941 | 40.5 |
| 19 | 80 | 326 | 36 | 4085 | 16.5 |
| 20 | 29.8 | 565.1 | 41.1 | 1712 | 67.8 |
| 21 | 40.4 | 440.9 | 20.7 | 2687 | 55.7 |
| 22 | 70.8 | 597.9 | 51 | 3268 | 68.1 |
| 23 | 105.3 | 193.2 | 43.8 | 978 | 40.8 |
| 24 | 29.9 | 223.4 | 32.8 | 1289 | 22.6 |
| 25 | 52.1 | 338.4 | 21.1 | 1567 | 35.3 |
| 26 | 140.3 | 787 | 46.4 | 12126 | 125 |
| 27 | 105.3 | 374 | 90.2 | 1211 | 227.1 |
| 28 | 55.1 | 580.9 | 37.9 | 2090 | 47.8 |
| 29 | 83.7 | 232 | 41.6 | 1083 | 127.7 |
| Mean | 102.5 | 520.7 | 49.9 | 2853.5 | 72.1 |
| S.E.M. | 21.8 | 32.7 | 3 | 510.8 | 18.9 |
| S.D. | 109.9 | 165.3 | 15.8 | 2617.3 | 95.9 |

Non-Responders

| | | | | | |
|---|---|---|---|---|---|
| 30 | 50.7 | 469.4 | 88.3 | 1111 | 491.7 |
| 31 | 215.4 | 768.5 | 62.2 | 554 | 59.1 |
| 32 | 142.2 | 710.2 | 79.1 | 3556 | 114.6 |
| 33 | 102.1 | 676.9 | 57.3 | 614 | 30.5 |
| 34 | 128.7 | 749.1 | 49.2 | 3189 | 165.2 |
| 35 | 196.3 | 561.1 | 68.5 | 1200 | 433.9 |
| 36 | 321.9 | 875.9 | 43.4 | 2242 | 494 |
| 37 | 155.4 | 864.8 | 66.5 | 2249 | 31 |
| 38 | 235.9 | 630.6 | 55.6 | 3261 | 375.1 |
| 39 | 107 | 638.9 | 65.7 | 1974 | 411.3 |
| 40 | 305.4 | 532.4 | 53.6 | 2772 | 83.2 |
| 41 | 431.4 | 558.3 | 73.1 | 255 | 34.8 |
| 42 | 455.3 | 601.4 | 75.1 | 260 | 134.8 |
| 43 | 150.2 | 712.1 | 80.1 | 664 | 120.7 |
| 44 | 198.8 | 580.2 | 72.6 | 1140 | 448.8 |
| 45 | 248.2 | 628.8 | 60.3 | 2382 | 380 |
| 46 | 118.5 | 699.2 | 55.3 | 1436 | 124.4 |
| 47 | 202.5 | 1277.1 | 90.2 | 1567 | 66.2 |
| 48 | 128.5 | 1051.1 | 64.8 | 1156 | 453.4 |
| 49 | 237.8 | 668.3 | 62.3 | 573 | 377.8 |
| 50 | 188 | 187 | 17.8 | 458 | 585 |
| 51 | 189.1 | 179 | 54.2 | 3755 | 614.8 |
| Mean | 205 | 664.6 | 63.4 | 1653.1 | 274.1 |
| S.E.M. | 20.5 | 54.5 | 4.1 | 216.2 | 39 |
| S.D. | 101.2 | 237.9 | 15.8 | 1121.1 | 201.4 |
| p | 0.001 | 0.02 | 0.004 | 0.032 | 0.000163 |

Table III: Using ELISA, the five differentially expressed protein concentrations were measured in duplicate for each patient. This table also shows the mean, standard error of the mean (SEM), standard deviation (SD) and the p-value for each protein.
B2M: beta-2-microglobulin,
VDB: Vitamin D binding protein,
ZAG: Zinc alpha 2-glycoprotein,
Hp: Haptoglobin,
SAA: Serum Amyloid A protein.

TABLE IV

| Patients | Patients | Clinical | Day-100 | Clinical | LOOCV | Predicted |
|---|---|---|---|---|---|---|
| Age | Sex | Stage ‘ISS’ | re-staging | Classification | P(NR) | Classification |
| Responders | | | | | | |
| 73 | F | III | VGPR | R | 0.259 | R |
| 66 | M | II | CR | R | 0.034 | R |
| 58 | F | III | CR | R | 0.264 | R |
| 67 | M | II | VGPR | R | 0.085 | R |
| 59 | M | III | VGPR | R | 0.692 | NR |
| 67 | M | III | CR | R | 0.021 | R |
| 72 | M | III | CR | R | 0.552 | NR |
| 60 | M | II | CR | R | 0.809 | NR |
| 71 | F | II | VGPR | R | 0.003 | R |
| 69 | M | I | VGPR | R | 0.345 | R |
| 61 | F | II | CR | R | 0.076 | R |
| 74 | F | I | VGPR | R | 0.017 | R |
| 70 | F | II | VGPR | R | 0.025 | R |
| 61 | F | I | VGPR | R | 0 | R |
| 77 | M | II | VGPR | R | 0.046 | R |
| 63 | M | I | CR | R | 0.092 | R |
| 58 | F | I | VGPR | R | 0.952 | NR |
| 59 | F | I | VGPR | R | 0.014 | R |
| 64 | M | II | CR | R | 0 | R |
| 63 | F | II | VGPR | R | 0.17 | R |
| 65 | M | II | VGPR | R | 0.011 | R |
| 70 | M | III | VGPR | R | 0.059 | R |
| 66 | F | II | CR | R | 0.002 | R |
| 80 | M | II | VGPR | R | 0.001 | R |
| 66 | F | II | VGPR | R | 0.006 | R |
| 81 | F | III | VGPR | R | 0 | R |
| 52 | M | III | VGPR | R | 0.36 | R |
| 64 | M | II | VGPR | R | 0.106 | R |
| 68 | F | II | VGPR | R | 0.013 | R |
| Non-Responders | | | | | | |
| 72 | F | II | PD | NR | 0.994 | NR |
| 74 | M | III | PD | NR | 0.878 | NR |
| 71 | M | III | PD | NR | 0.161 | R |
| 65 | M | II | PD | NR | 0.519 | NR |
| 70 | M | III | PD | NR | 0.674 | NR |
| 63 | F | III | PD | NR | 0.995 | NR |
| 72 | M | III | PD | NR | 1 | NR |
| 63 | M | II | SD | NR | 0.7 | NR |
| 78 | M | II | PD | NR | 0.951 | NR |
| 79 | F | II | PD | NR | 0.994 | NR |
| 57 | F | III | PD | NR | 0.01 | R |
| 77 | M | III | PD | NR | 0.19 | R |
| 78 | F | II | PD | NR | 0.791 | NR |
| 64 | F | II | PD | NR | 0.902 | NR |
| 65 | F | I | SD | NR | 0.997 | NR |
| 73 | F | III | PD | NR | 0.981 | NR |
| 78 | M | II | SD | NR | 0.786 | NR |
| 61 | F | II | SD | NR | 1 | NR |
| 76 | F | III | PD | NR | 1 | NR |
| 77 | M | II | PD | NR | 0.998 | NR |
| 62 | M | I | PD | NR | 0.974 | NR |
| 69 | M | II | SD | NR | 0.336 | R |

Table IV: This table outlines the clinical details of the patients included in this study, including their age, sex, clinical stage based on ISS (International Staging System), Day-100 restaging based on IMWG (International Myeloma Working Group) uniform response criteria for multiple myeloma and clinical classification of response to thalidomide. The last two columns summarize the leave-one-out cross validation (LOOCV) analysis. If the resulting value of p is below 0.5, response to thalidomide is predicted and if the value is above 0.5 non-response to thalidomide is predicted.

TABLE V

| Patients | Patients | Clinical | Day-100 | Clinical | Induction | 2nd Line | Follow | |
|---|---|---|---|---|---|---|---|---|
| Age | Sex | Stage ‘ISS’ | re-staging | Classification | Therapy | Treatment | up months | Current status |
| Responders | | | | | | | | |
| 73 | F | III | VGPR | R | MPT | N/A | 17 | VGPR |
| 58 | F | III | CR | R | TD | SCT | 16 | CR |
| 67 | M | II | VGPR | R | TD | N/A | 12 | VGPR |
| 59 | M | III | VGPR | R | TD | BORT & SCT | 18 | VGPR |
| 67 | M | III | CR | R | TD | LEN | 16 | CR |
| 72 | M | III | CR | R | TD | N/A | 13 | CR |
| 60 | M | II | CR | R | TD | SCT | 19 | CR |
| 71 | F | II | VGPR | R | CTD | N/A | 13 | VGPR |
| 69 | M | I | VGPR | R | TD | N/A | 12 | VGPR |
| 61 | F | II | CR | R | TD | SCT | 19 | CR |
| 74 | F | I | VGPR | R | TD | BORT | 13 | VGPR |
| 70 | F | II | VGPR | R | TD | BORT | 14 | CR |
| 61 | F | I | VGPR | R | TD | SCT | 12 | VGPR |
| 77 | M | II | VGPR | R | MPT | N/A | 14 | VGPR |
| 63 | M | I | CR | R | TD | N/A | 14 | CR |
| 59 | F | I | VGPR | R | TD | N/A | 14 | SCT |
| 64 | M | II | CR | R | CTD | N/A | 12 | CR |
| 63 | F | II | VGPR | R | TD | BORT & SCT | 21 | VGPR |
| 65 | M | II | VGPR | R | TD | SCT | 8 | VGPR |
| 70 | M | III | VGPR | R | TD | N/A | 7 | VGPR |
| 66 | F | II | CR | R | CTD | N/A | 8 | VGPR |
| 80 | M | II | VGPR | R | TD | N/A | 7 | VGPR |
| 66 | F | II | VGPR | R | TD | BORT | 10 | VGPR |
| 81 | F | III | VGPR | R | TD | N/A | 6 | VGPR |
| 52 | M | III | VGPR | R | TD | SCT | 11 | CR |
| 64 | M | II | VGPR | R | TD | Awaiting SCT | 6 | VGPR |
| Non-Responders | | | | | | | | |
| 72 | F | II | PD | NR | TD | BORT | 16 | VGPR |
| 74 | M | III | PD | NR | MPT | BORT + LEN | 12 | VGPR |
| 71 | M | III | PD | NR | TD | LEN | 19 | CR |
| 65 | M | II | PD | NR | TD | BORT + SCT | 11 | CR |
| 70 | M | III | PD | NR | CTD | LEN | 17 | VGPR |
| 63 | F | III | PD | NR | TD | BORT + SCT | 14 | VGPR |
| 72 | M | III | PD | NR | CTD | BORT | 16 | NR |
| 63 | M | II | SD | NR | TD | LEN | 11 | VGPR |
| 78 | M | II | PD | NR | CTD | LEN | 10 | NR |
| 79 | F | II | PD | NR | TD | VEL | 15 | VGPR |
| 57 | F | III | PD | NR | TD | VEL + SCT | 10 | VGPR |
| 77 | M | III | PD | NR | TD | REFUSE | 12 | RIP |
| 78 | F | II | PD | NR | TD | LEN | 15 | VGPR |
| 64 | F | II | PD | NR | TD | VEL + SCT | 21 | VGPR |
| 65 | F | I | SD | NR | TD | VEL | 20 | NR |
| 73 | F | III | PD | NR | CTD | VEL + LEN | 18 | VGPR |
| 78 | M | II | SD | NR | TD | VEL | 11 | NR |
| 61 | F | II | SD | NR | TD | VEL & SCT | 12 | NR |
| 76 | F | III | PD | NR | TD | VEL | 9 | NR |
| 77 | M | II | PD | NR | TD | LEN | 11 | VGPR |
| 62 | M | I | PD | NR | TD | VEL | 8 | VGPR |
| 69 | M | II | SD | NR | TD | LEN | 6 | PD |

Table V: This table outlines the clinical details of the patients included in this study, including their age, sex, clinical stage based on ISS (International Staging System), Day-100 restaging based on IMWG (International Myeloma Working Group) uniform response criteria for multiple myeloma and clinical classification of response to thalidomide. Also included in this table are details for thalidomide-based induction regiment, second line treatment, duration of follow-up in months and the current clinical status.
Abbreviations:
CR: complete response,
VGPR: very good partial response,
SD: stable disease,
PD: progressive disease,
IMWG: International Myeloma Working Group,
R: Responders to thalidomide,
NR: Non-responders to thalidomide,
BORT: Bortezomib,
LEN: Lenalidomide,
SCT: Stem Cell Transplant,
M: male,
F: female,
N/A: Not applicable,
RIP: Rest in Peace.
TD: thalidomide and dexamethasone,
CTD: cyclophosphamide, thalidomide, and dexamethasone,
MPT: melphalan, prednisone and thalidomide

The invention is not limited to the embodiments hereinbefore described which may be varied in construction and detail without departing from the spirit of the invention.

1-17. (canceled)

18. A method for predicting an individuals response to thalidomide or a thalidomide analog as a treatment for multiple myeloma, the method comprising a step of assaying a biological sample from the individual to determine the abundance of a panel of biomarkers comprising SAA and VDB, and at least one of ZAG, Hp and B2M, and comparing the abundance value for each biomarker with a reference abundance value for each biomarker from a Responder or Non-responder, and correlating the differential abundance for the biomarkers to predict the individuals response.

19. A method as claimed in claim 18 which the panel of biomarkers comprises SAA, VDB and ZAG, and optionally one or more biomarkers selected from B2M and Hp.

20. A method as claimed in claim 18 in which the panel of biomarkers comprises SAA, VDB and Hp, and optionally one or more biomarkers selected from ZAG and B2M.

21. A method as claimed in claim 18 in which the panel of biomarkers comprises SAA, VDB and B2M, and optionally one or more biomarkers selected from ZAG and Hp.

22. A method as claimed in claim 18 in which the abundance value for each biomarker is compared with a reference abundance value for each biomarker from a Responder.

23. A kit of parts comprising diagnostic reagents capable of quantitative detection of a panel of biomarkers comprising VDB and SAA, and at least one of ZAG, Hp and B2M, and instructions for the use of the reagents in determining the response of an individual with multiple myeloma, to thalidomide or a thalidomide analog.

24. A kit of parts as claimed in claim 23 in which the or each diagnostic reagent is selected from an antibody, or antibody fragment, capable of specifically binding to the target biomarker

25. A kit of parts as claimed in claim 23 including diagnostic reagents for the quantitative detection of a panel of biomarkers comprising SAA, VDB and ZAG, and optionally one or more biomarkers selected from B2M and Hp.

26. A kit of parts as claimed in claim 23 including diagnostic reagents for the quantitative detection of a panel of biomarkers comprising SAA, VDB and Hp, and optionally one or more biomarkers selected from ZAG and B2M.

27. A kit of parts as claimed in claim 23 including diagnostic reagents for the quantitative detection of a panel of biomarkers comprising SAA, VDB and B2M, and optionally one or more biomarkers selected from ZAG and Hp.

28. An immunoassay kit comprising a support having affixed thereon an antibodies, or antibody fragments, capable of specifically binding to a panel of biomarkers comprising VDB and SAA, and at least one of ZAG, B2M, and Hp, and means for quantitatively detecting specific binding between the antibodies, or antibody fragments, and the biomarkers proteins.

29. An immunoassay kit as claimed in claim 28 and comprising a support having affixed thereon an antibodies, or antibody fragments, capable of specifically binding to a panel of biomarkers comprising VDB, SAA and ZAG, and optionally one or more of B2M and Hp.

30. An immunoassay kit as claimed in claim 28 and comprising a support having affixed thereon an antibodies, or antibody fragments, capable of specifically binding to a panel of biomarkers comprising VDB, SAA and B2M, and optionally one or more of ZAG and Hp

31. An immunoassay kit as claimed in claim 28 and comprising a support having affixed thereon an antibodies, or antibody fragments, capable of specifically binding to a panel of biomarkers comprising VDB, SAA and Hp, and optionally one or more of B2M and ZAG.

32. An immunoassay kit as claimed in claim 28 in the form of an ELISA kit.

33. A kit of parts as claimed in claim 23.

34. A method for predicting an individuals response to thalidomide or a thalidome analog as a treatment for multiple myeloma, the method comprising a step of assaying a biological sample from the individual to determine a differential abundance for each of a panel of biomarkers comprising SAA and VDB, and at least one of ZAG, Hp and B2M, and correlating the differential abundances to predict the individuals response to thalidomise or a thalidomide analog, wherein the differential abundance for a biomarker is the difference between the abundance in the individual and a reference abundance for a Responder or Non-responder.

35. An immunoassay kit as claimed in claim 28, for use in predicting an individuals response to thalidomide or a thalidomide analog as a treatment for multiple myeloma.
